(19) United States
(12) Patent Application Publication
(10) Pub. No.: US 2019/0167822 A1
(43) Pub. Date: Jun. 6, 2019

(54) NON-IODINATED RADIOLABELED RADIOPAQUE MICROBEADS WITH MRI CONTRAST FOR RADIOEMBOLIZATION

(71) Applicants: Manzoor KOYAKUTTY, Kochi, Kerala (IN); Vijay Harish SOMASUNDARAM, Kochi, Kerala (IN); Anusha ASHOKAN, Kochi, Kerala (IN); Shantikumar NAIR, Kochi, Kerala (IN); AMRITA VISHWA VIDYAPEETHAM, Kochi, Kerala (IN)

(72) Inventors: Manzoor KOYAKUTTY, Kochi (IN); Vijay Harish SOMASUNDARAM, Kochi (IN); Anusha ASHOKAN, Kochi (IN); Shantikumar NAIR, Kochi (IN)

(21) Appl. No.: 16/302,565

(22) PCT Filed: May 19, 2017
(86) PCT No.: PCT/US17/33486

§ 371 Date: Nov. 16, 2018

(30) Foreign Application Priority Data
May 19, 2016 (IN) 201641017865

(51) Int. Cl. A61K 51/12; A61K 51/04; A61P 35/00

(52) U.S. Cl. CPC A61K 51/1251; A61K 45/06; A61P 35/00; A61K 51/0474

(57) ABSTRACT

The invention discloses non-iodinated radiopaque microbeads that may be used in image guided embolization in a subject ailing with tumor. The non-iodinated radiopaque microbeads include a ceramic material doped with a CT contrast agent or a MRI contrast agent or both. The doped ceramic is blended with a polymer and the blend is electrosprayed to form the radiopaque microbeads. Further the radiopaque microbeads are radiolabeled with a radioactive isotope. Methods of synthesis of the radiopaque microspheres are also disclosed. The non-iodinated radiopaque microbeads with radiolabeling are capable of rendering an imageable computed tomography (CT) contrast or magnetic resonance imaging (MRI) contrast when administered in a subject. Also the microspheres are biodegradable and hence the treatment could be repeated in case of recurrence of the tumor in the subject.

### CROSS-REFERENCES TO RELATED APPLICATIONS

This application is a national phase application of PCT application No. PCT/US17/33486 filed on May 19, 2017, which claims priority to Indian Provisional Application No. 201641017865 filed on May 19, 2016, the full disclosure of which is incorporated herein by reference.

### FIELD OF THE INVENTION

The invention is related to microbead composition and in particular to a radiopaque microsphere for use in image guided embolization in a subject.

### BACKGROUND

Image-guided transarterial embolization is a therapy for the embolization of malignant tumors in organs and arteriovenous malformations. Embolizing agents include viscous liquids, particulate materials and mechanical devices. Recently microspheres or microbeads are included in this list. The microbeads are mixed with a soluble contrast agent during administration in a subject, to aid temporary visualization of the site of delivery. The success of embolization is determined by the absence of flow of the soluble contrast agent beyond the site of desired embolization. The actual location of these embolizing beads is unknown and is inferred only using indirect and temporary signs. Thus, there is a need for inherently radiopaque embolizing agents, that could directly assess completeness of target tissue embolization and also could help identify non-target sites that may have been accidentally embolized during the procedure.

Further, such radiopaque beads allow follow up assessment of the patients without need of additional soluble (iodinated) contrast administration. Transarterial radioembolization (TARE), is an advancement in transarterial embolization technique where microbeads are blended with a therapeutic radioisotope (primarily β particle emitters) to have a localized radiation mediated therapeutic effect on tumors, besides the effect caused by embolization of the tumor arteries. The presently used microbeads for TARE are either glass or resin based, and again not inherently radiopaque. Further, they are non-biodegradable hence prevent retreatment with a second sitting of TARE (as the blood vessels are permanently blocked) to treat any residual or recurrent tumors at the same site.

Another shortcoming of these presently used radioactive microbeads is that the radioisotopes are embedded within the beads during the manufacture process, hence incurring the additional cost during manufacturing and in ensuring radiation safety during the shipment of each batch of microbeads. The invention is oriented toward solving some of the above problems in existing materials and techniques

### SUMMARY OF THE INVENTION

In various embodiments a non-iodinated, radiopaque microbead or microsphere composition for use in image-guided embolization or radioembolization in a subject, includes a ceramic material (C), that includes an impurity dopant (X), wherein the impurity dopant X is present at a concentration of 0-30% (w/w) and is selected from molybdenum, tungsten, zirconium or gold or a combination thereof. The composition further includes a polymer binder blended to the ceramic material and a radioisotope conjugated to the ceramic material or the microbeads. The composition may render an imageable computed tomography (CT) contrast or magnetic resonance imaging (MRI) contrast, or both, when administered to the subject.

In some embodiments the imaged-guided embolization is selected from transarterial embolization (TE), transarterial radioembolization (TARE) or Selective Internal Radiation Therapy (SIRT) of tumors. In some embodiments the size of the microsphere is 1-1500 μm. In some embodiments the composition may include a magnetic resonance imaging (MRI) contrast agent or dopant, doped or loaded in the ceramic material.

In some embodiments the MRI contrast agent is selected from iron, manganese, terbium, erbium, dysprosium, holmium, thulium, bismuth, barium, strontium, iodine, zirconium, lanthanides, hafnium or aluminum. In various embodiments the ceramic material is selected from the group consisting of alumina, zirconia, silica, hydroxyapatite, Calcium aluminate, bioactive glass, cerium oxide, calcium sulphate, calcium molybdate, calcium silicates, calcium carbonate, α-tricalcium phosphate, β-tricalcium phosphate, octocalcium phosphate, dicalcium phosphates, tetracalcium phosphate monoxide, ferric-calcium-phosphorus oxides, Biocorals or any combination thereof.

In various embodiments the polymer is selected from the group comprising of polyvinyl alcohols, polyacrylic acids, polymethacrylic acids, polyethylenimines, poly vinyl sulphonates, carboxymethyl celluloses, hydroxymethyl celluloses, oxidized cellulose, gellan, gum arabic, substituted celluloses, polyanhydrides, poly ortho esters, polyacrylamides, polyethylene glycols, polyamides, polyvinylpyrrolidones, polyureas, polyurethanes, polyesters, polyethers, polystyrenes, polysaccharides, polylactic acids, polyethylenes, polymethyl methacrylates, polycaprolactones, polyvinyl acetate, polyglycolic acids, poly(lactic-co-glycolic) acids, albumin, transferrin, caseins, gelatin, mannose, sucrose, starch, galactose, galactomannans, or a combination thereof.

In various embodiments the polymer may be a biopolymer selected from alginate, gelatin, collagen, chitosan, carboxymethyl chitosan, chitin, cellulose, carboxymethyl cellulose, dextran, fibrin, hyaluronic acid, chondroitin sulphate, agarose, starch, poly[lactic-co-glycolic] acid, poly-L-lactic acid, polylactic acid, polycaprolactone, polyvinyl alcohol, polyhydroxy butyrate, polyhydroxybutyrate-co-hydroxyvalerate, polyphosphazenes, polyurethane, or polyanhydrides. In some embodiments composition of claim 1, wherein the polymer is present at a concentration of 0-30% (w/w) of the ceramic.

In various embodiments the microspheres are biodegradable and undergo degradation in at least two months. In various embodiments the composition is administered by intra-arterial, transarterial, intra-articular or local route. In various embodiments the microspheres are non-iodinated and show combinatorial CT and MRI contrast in T2 mode with relaxivity of at least R2=17 mM−1s−1. In various embodiments the radioisotope is selected from 99mTc, 111In, 123I, 131I, 188Re, 186Re, 166Ho, 32P, 18F, 68Ga, 177Lu, 90Y, 166Dy, 103Pd, 169Yb, 212Bi, 213Bi, 212Po, 225Ac, 211At, 89Sr, 192Ir, 1941r or 223Ra conjugated to the microspheres.

In various embodiments a method of synthesis of radiopaque microsphere or microbeads capable of rendering an imageable computed tomography (CT) contrast when administered in a subject includes doping or co-loading an impurity dopant in a ceramic to obtain a ceramic material. The impurity dopant is present at a concentration of 0-30% (w/w) and is selected from molybdenum, tungsten or zirconium or a combination thereof, for X ray contrast and from iron, manganese, terbium, erbium, dysprosium, holmium, thulium, bismuth, barium, strontium, iodine, zirconium, lanthanides, hafnium or aluminium or combinations thereof, for MRI contrast. The method further includes blending the ceramic material with a polymer solution, electrospraying the blend with or without a crosslinker to form microbeads, incubating the formed microbeads at an optimum temperature for 48 hours and radiolabeling the microbeads with a radioisotope to produce the radiolabeled radiopaque microbeads.

In some embodiments the crosslinker is selected from one of bivalent cationic solutions comprising calcium chloride, stannous chloride, barium chloride or ferric chloride, carbodiamides, EDC, trivinyl sulphones, acrylamides, epoxides, polyamides, maleimide, iminoesters, or combinations thereof. In some embodiments the method further includes the step of calcining the beads in the temperature range 100-1000° C. to produce the radiopaque beads. In various embodiments the beads are further subjected to lyophilization to produce the radiopaque beads. In some embodiments the synthesized microbeads are non-iodinated. In various embodiments the radiolabeling of microbeads is done either by direct interaction or by using an appropriate ligand or chelating agent.

In various embodiments the ligand or chelating agent is selected from bisphosphonates, DMSA, DMDTPA, ethylene dicysteine, mercaptoacetyltriglycine, hydrazinonicotinamide, iminodiacetic acid, a crown ether, DTPA monoamide, EDTA, DOTA, EGTA, BAPTA, DO3A, NOTA-Bn, styrene, butyl acrylate, glycidil methacrylate, aminocarboxylic acids, NODASA, NODAGA, peptides, oligomers, amino acids, 10-decanedithiol (HDD), ethyl cysteinate dimer complexes, DEDC, methoxyisobutylisonitrile, or derivatives or combinations thereof. In some embodiments the ligand or chelating agent, with or without radiolabeling, is either covalently or electrostatically bound to the microbeads. In various embodiments the method further includes conjugating the microsphere with a chemotherapeutic agent.

In various embodiments a method of medical treatment of a tumor in a mammal is disclosed. The method includes administering a therapeutically effective amount of a non-iodinated, radiopaque microbead or microsphere composition for use in image-guided embolization or radioembolization of the tumor. The microsphere includes a ceramic material (C), comprising an impurity dopant (X), wherein the impurity dopant is present at a concentration of 0-30% (w/w) and is selected from molybdenum, tungsten, zirconium or gold or a combination thereof. A polymer binder is blended to the ceramic material, and a radioisotope is conjugated to the ceramic material or the microbeads, wherein the composition renders imageable computed tomography (CT) contrast or magnetic resonance imaging (MRI) contrast, or both, when administered to the subject.

In some embodiments the non-iodinated radiopaque microbeads are used in combination with radiofrequency ablation, chemotherapy or immunotherapy. In some embodiments the non-iodinated radiopaque microbeads are biodegradable. In various embodiments the method is repeated in the subject on re-occurrence of the tumor.

This and other aspects are disclosed herein

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1A illustrates the radiopaque microbead composition with radiolabeling.

FIG. 1B shows the radiopaque microbead composition that includes ceramic material, a CT contrast dopant, a MRI contrast dopant blended with a polymer.

FIG. 1C shows the ceramic material doped with a CT contrast impurity to form a radiopaque ceramic material.

FIG. 1D illustrates the formation of radiopaque microbead with radiolabeling.

FIG. 2 illustrates the method of synthesis of radiopaque microbead with radiolabeling formed from impurity doped ceramic.

FIG. 3A shows the preparation of molybdenum doped calcium phosphate and calcium molybdate to demonstrate the radiopacity rendered by using different dopant concentrations.

FIG. 3B illustrates the variation in radiopacity rendered by using different dopant concentrations.

FIG. 3C illustrates successful doping and chemical composition of the doped ceramics using ICP, XRD technique.

FIG. 3D illustrates successful doping and chemical composition of the doped ceramics using EDX technique.

FIG. 4A shows the electrospraying of molybdenum doped in calcium phosphate and blended with sodium alginate solution to form microbeads.

FIG. 4B shows radiopaque microbeads of size ranging between 200-300 μm.

FIG. 4C shows radiopaque microbeads of size ranging between 300-500 μm.

FIG. 4D shows radiopaque microbeads of size ranging between 500-800 μm.

FIG. 5A illustrates the stereomicroscopic images of the radiopaque microbeads demonstrating their spherical shape.

FIG. 5B illustrates the stereomicroscopic images of the radiopaque microbeads showing their pliable nature when moist.

FIG. 6A shows the experimental set-up, where the said microbeads are contained at the bottom of a centrifugation tube with the Tc-MDP as a clear solution above it.

FIG. 6B shows the nuclear image of the tube 15 minutes after incubation at room temperature.

FIG. 6C shows the nuclear image of the tube 1.5 hours after incubation at room temperature.

FIG. 7A illustrates the CT images of the agar phantoms containing the radiolabeled microbeads, showing the distinct radiopacity of the microbeads.

FIG. 7B shows the SPECT images of the phantom, indicating the site of the nuclear signal within it.

FIG. 7C shows the fused SPECT & CT images localizing the nuclear signal to the radiopaque microbeads that had been radiolabeled.

FIG. 8A illustrates the X-ray images of a New Zealand white (NZW) rabbit with the radiopaque microbeads implanted subcutaneously (indicated by black arrow) to study their degradation in vivo after 1 week of transplant.

FIG. 8B shows the image acquired at 1 month post implantation, that show intact radiopacity.

FIG. 8C shows the image acquired at 2 months post implantation that show reduction in intensity of radiopacity indicating degradation of the microbeads.

FIG. 9A shows the X-ray angiogram image before embolization of the right renal artery (arrow) clearly showing the contrast entering the renal artery and into its branches in the kidney.

FIG. 9B illustrates the post-embolization image showing there is absolutely no contrast entering the right renal arterial system, indicating complete arterial blockage by the microbeads.

FIG. 10A illustrates the X-ray image of post-embolization of the renal artery using the radiopaque microbeads, arrow on inset image indicated the radiopacity retained within the renal arterial system.

FIG. 10B shows the H&E stained histological slide of the embolized kidney which confirms the presence of the microbeads blocking the renal artery and its branches (black arrow).

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The present invention in various embodiments relates to microbead composition and in particular to radiopaque microspheres that may be used in image guided embolization in a subject. Further, a method of synthesis of the radiopaque microspheres, that are capable of rendering an imageable computed tomography (CT) contrast along with radiolabeled contrast when administered in a subject is disclosed.

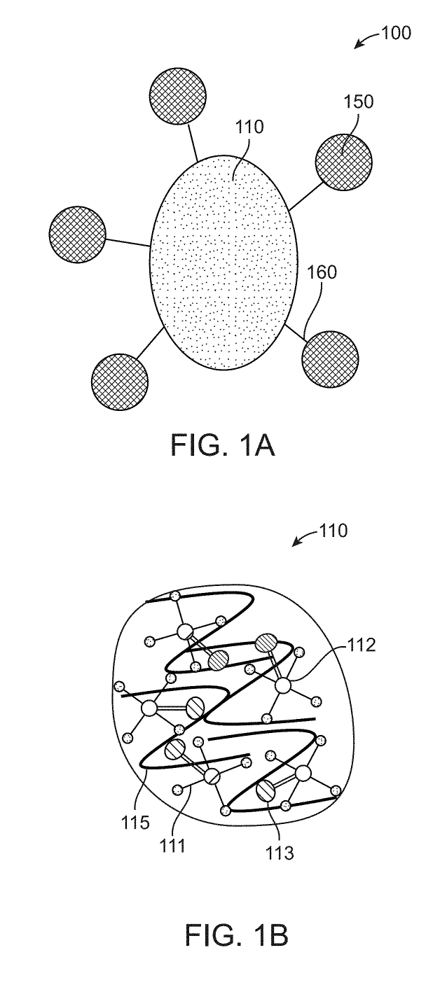

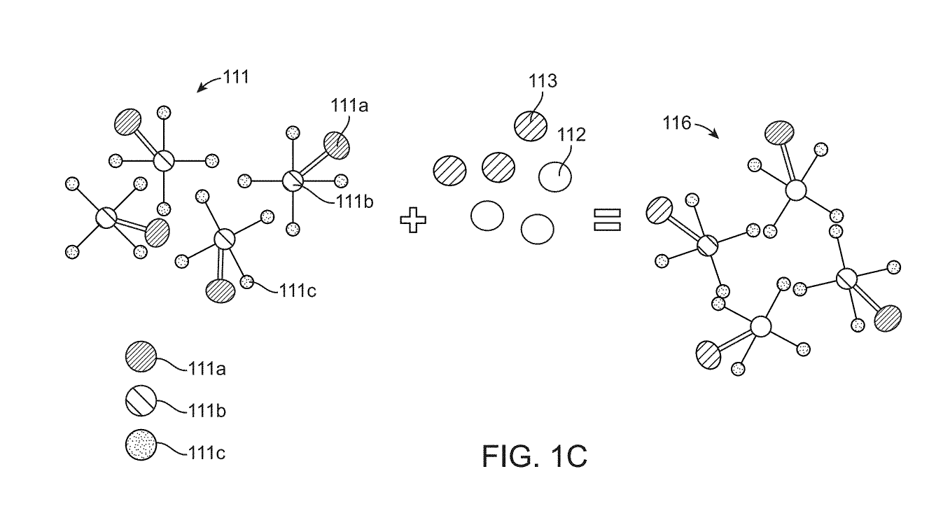

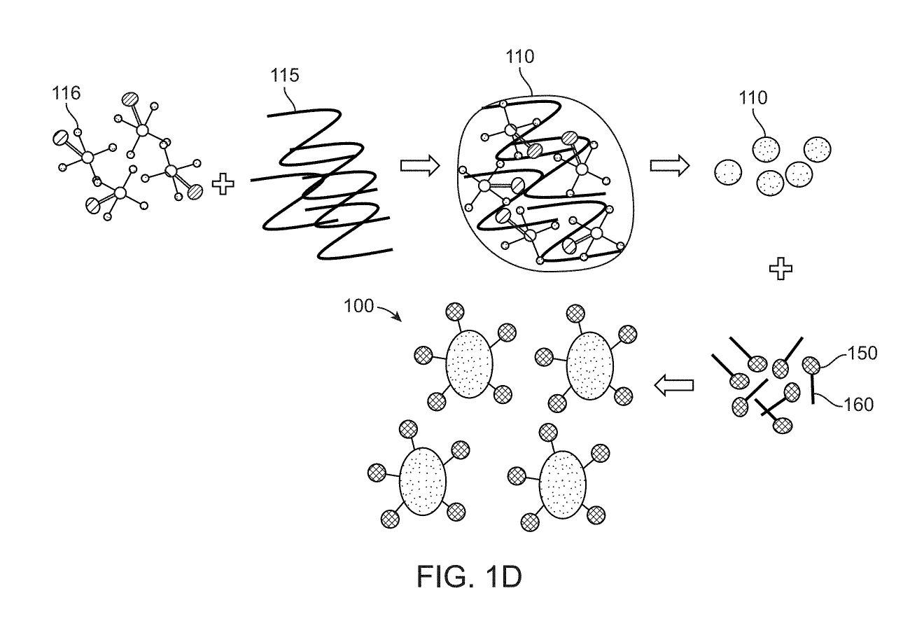

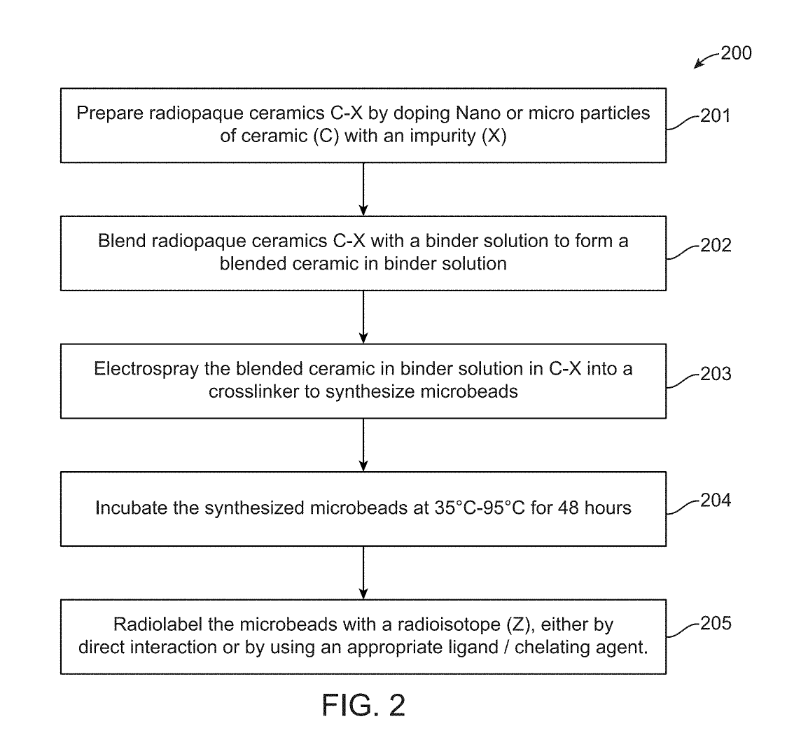

In various embodiments the non-iodinated radiopaque microsphere composition with radiolabeling 100 as shown in FIG. 1A includes a radiopaque microsphere 110 and a radioactive isotope 150 conjugated with the radiopaque microsphere 110. The radiopaque microsphere 110 as shown in FIG. 1B includes a ceramic material 111 that includes one or more impurity dopant 112, 113. A polymer 115 is then blended with the radiopaque ceramic material 111 to form a blended ceramic in binder. The blended ceramic in binder are then electrosprayed to form the radiopaque microsphere composition 110. The impurity dopant 112 is configured to provide radiopacity and CT contrast to the ceramic material and the impurity dopant 113 is configured to provide MRI contrast. The impurity dopants 112, 113 are present at a concentration of 0-30% (w/w). A radioisotope 150 is then conjugated with the radiopaque microsphere 110 as shown in FIG. 1A. The radiopaque microsphere composition with radiolabeling 100 may be used in image guided embolization in a subject that may render an imageable computed tomography (CT) or magnetic resonance imaging (MRI) contrast when administered to the subject.

In various embodiments the ceramic material 111 may include one or more elements as shown in FIG. 1C. The elements may include calcium 111a, sulphur 111b and oxygen 111c. Further one or more dopants 112 and 113 are added to the ceramic material 111 to form a radiopaque ceramic material 116. Dopant 112 is configured to provide radiopacity to the ceramic material 111. Dopant 112 may include a CT contrast agent that may also provide CT contrast to the ceramic material 111. Dopant 113 may include a MRI contrast agent that is configured to provide MRI contrast to the radiopaque ceramic material.

In various embodiments the ceramic material 111 may include one or more of alumina, zirconia, silica, hydroxyapatite, calcium aluminate, bioactive glass, cerium oxide, calcium sulphate, calcium molybdate, calcium silicates, calcium carbonate, calcium phosphates, α-tricalcium phosphate, β-tricalcium phosphate, octocalcium phosphate, dicalcium phosphates, tetracalcium phosphate monoxide, ferric-calcium-phosphorus oxides or Bio-corals.

In various embodiments the impurity dopant 112 that is doped with the ceramic material 111 to form the radiopaque ceramic material 116 as shown in FIG. 1C may include one or more CT contrast agent selected from molybdenum, tungsten, zirconium, or gold.

In some embodiments the radiopaque microsphere composition 110 is either doped or loaded with an MRI contrast agent 113 to enable temporary visualization of the site of delivery of the microsphere. In various embodiments the MRI contrast agent 113 that is doped with the microsphere composition is selected from one or more of iron, manganese, terbium, erbium, dysprosium, holmium, thulium, bismuth, barium, strontium, iodine, zirconium, lanthanides, hafnium or aluminium.

In some embodiments a polymer 115 as shown in FIG. 1D is blended to the radiopaque ceramic material 116 to form the polymer blended ceramic in binder 110 as shown in FIG. 1D. The polymer 115 may be present at a concentration of 0-30% weight of the ceramic material. The polymer 115 may include one or more of polyvinyl alcohols, polyacrylic acids, polymethacrylic acids, polyethyleneimine, poly vinyl sulphonates, carboxymethyl celluloses, hydroxymethyl celluloses, oxidized cellulose, gellan, gum arabic, substituted celluloses, polyanhydrides, poly (ortho)esters, polyacrylamides, polyethylene glycols, polyamides, polyvinylpyrrolidones, polyureas, polyurethanes, polyesters, polyethers, polystyrenes, polysaccharides, polylactic acids, polyethylenes, polymethyl methacrylates, polycaprolactones, polyvinyl acetate, polyglycolic acids, poly(lactic-co-glycolic) acids, albumin, transferrin, caseins, gelatin, mannose, sucrose, starch, galactose, or galactomannans.

In some embodiments the polymer 115 that is blended to the ceramic material may include one or more biopolymers including alginate, gelatin, collagen, chitosan, carboxymethyl chitosan, chitin, cellulose, carboxymethyl cellulose, dextran, fibrin, hyaluronic acid, chondroitin sulphate, agarose, starch, poly[lactic-co-glycolic] acid, poly-L-lactic acid, polylactic acid, polycaprolactone, polyvinyl alcohol, polyhydroxy butyrate, polyhydroxy butyrate co hydroxyvalerate, polyphosphazenes, polyurethane, or polyanhydrides.

In some embodiments the radioisotope 150 as shown in FIG. 1D is conjugated with the radiopaque ceramic material 110 to form the radiolabeled radiopaque microbeads composition 100. In some embodiments a ligand or chelating agent 160 is configured to bind the radioisotope to the radiopaque microbeads 110. In various embodiments the radioisotope 150 may include one or more of ^{99m}Tc, ¹¹¹In, ¹²³I, ¹³¹I, ¹⁸⁸Re, ¹⁸⁶Re, ¹⁶⁶Ho, ³²P, ¹⁸F, ⁶⁸Ga, ¹⁷⁷Lu, ⁹⁰Y, ¹⁶⁶Dy, ¹⁰³Pd, ¹⁶⁹Yb, ²¹²Bi, ²¹³Bi, ²¹²Po, ²²⁵Ac, ²¹¹At, ⁸⁹Sr, ¹⁹²Ir, ¹⁹⁴Ir or ²²³Ra.

The radiolabeled radiopaque microsphere composition 110 in some embodiments may be used in imaged-guided embolization that may include transarterial embolization (TE), transarterial radioembolization (TARE) or Selective Internal Radiation Therapy (SIRT) of tumors.

In various embodiments the size of the radiopaque microspheres ranges from 1-1500 μm. In various embodiments the radiopaque microspheres are biodegradable and may degrade within 2 months of their administration in the subject. Biodegradability of the microspheres may allow clearance of the blocked blood vessel that leads to the tumor. This may allow re-treatment of residual or recurrent tumors with another sitting of TARE.

In some embodiments the radiopaque microsphere composition is administered in the subject is done under image guidance through any of intra-arterial, transarterial, intra-articular or local routes.

In some other embodiments the radiolabeled radiopaque microsphere composition are non-iodinated and show combinatorial CT and MRI contrast. Hence these non-iodinated radiopaque microsphere embolizing agents may allow direct assess of the target tissue embolization by image guidance and may also help identify non-target sites that may have been accidentally embolized during the procedure. Further, the radiolabeled radiopaque microbeads will also allow follow up assessment of the patients without need of additional iodinated soluble contrast administration.

In some embodiments a chemotherapeutic agent or an immunotherapy agent may be conjugated with the radiopaque microsphere composition and could be used in TA and TARE procedures. In some embodiments the chemotherapeutic agent is selected from one or more of doxorubicin, daunorubicin, temozolomide, carmustine, cisplatin, paclitaxel, curcumin or small molecule tyrosine kinase inhibitors.

In some other embodiments the radiopaque microspheres are pliable when moist and may be compressed to at least to 70% of the initial diameter. This property ensures that the microspheres can easily pass through small-bore catheters without breakage. In various embodiments the radiopaque microspheres may attenuate X-ray in the range 30-3000HU and in various embodiments may emit one or more of α, β, or γ radiation.

In various embodiments the invention includes a method of synthesis 200 of radiopaque microsphere or microbeads. The radiopaque microsphere or microbead is capable of rendering an imageable computed tomography (CT) contrast when administered in a subject. In various embodiments the method includes the following steps. In step 201 an impurity dopant (X) is either doped or co-loaded in a ceramic (C) to obtain a ceramic material (C-X). The impurity dopant (X) is present at a concentration of 0-30% (w/w) and may include one or more of molybdenum, tungsten, or zirconium. In step 202 the ceramic material is blended with a binder solution to form a blended binder solution. The binder solution may include a polymer solution. The blended binder solution is electrosprayed into a crosslinker in step 203 to form microspheres or microbeads. Further the formed microbeads in step 203 are incubating in step 204 at a temperature range of 35° C.-95° C. for 48 hours. In step 205 the incubated microbeads are radiolabeled with a radioisotope to produce the radiopaque microbeads.

In various embodiments the binder solution that forms a blend of the ceramic material may include one or more of polyvinyl alcohols, polyacrylic acids, polymethacrylic acids, polyethylenimines, poly vinyl sulphonates, carboxymethyl celluloses, hydroxymethyl celluloses, substituted celluloses, polyanhydrides, poly (ortho)esters, polyacrylamides, polyethylene glycols, polyamides, polyvinylpyrrolidones, polyureas, polyurethanes, polyesters, polyethers, polystyrenes, polysaccharides, polylactic acids, polyethylenes, polymethyl methacrylates, polycaprolactones, polyvinyl acetate, polyglycolic acids, poly(lactic-co-glycolic) acids, albumin, transferrin, caseins, gelatin, mannose, sucrose, starch, galactose, galactomannans, or a combination thereof.

In some embodiment the crosslinker may include one or more of bivalent cationic solutions comprising calcium chloride, stannous chloride, barium chloride or ferric chloride, carbodiamides, EDC, trivinyl sulphones, acrlyamides, epoxides, polyamides, maleimide, or iminoesters.

In some embodiments the method may include the step of annealing or calcining the microspheres in the temperature range 100-1000° C. to produce the radiopaque beads. In some embodiments the microspheres could be lyophilized.

In various embodiments the radiolabeling of microbeads are done with appropriate clinically approved radioisotopes that have a radiolabeling efficiency of ≥85%. In some embodiments the clinically approved radioisotopes may include one or more of ¹⁸⁸Rhenium, ^{99m}Tc, ¹¹¹In, ¹²³I, ¹³¹I, ¹⁸⁸Re, ¹⁸⁶Re, ¹⁶⁶Ho, ³²P, ¹⁸F, ⁶⁸Ga, ¹⁷⁷Lu, ⁹⁰Y, ¹⁶⁶Dy, ¹⁰³Pd, ¹⁶⁹Yb, ²¹²Bi, ²¹³Bi, ²¹²Po, ²²⁵Ac, ²¹¹At, ⁸⁹Sr, ¹⁹²Ir, ¹⁹⁴Ir or ²²³Ra. In some embodiments the radiolabeling may be performed either by direct interaction or by using an appropriate ligand or chelating agent. In some embodiments the chelating agent are either covalently or electrostatically bound to the microsphere during their preparation. A major advantage of the disclosed embodiments is that the radiopaque, biodegradable microbeads, can efficiently be radiolabeled in-house within the hot-lab at any nuclear medicine department. This will allow readily available radioactive microbeads of any desired dosage, which can be specifically tailored by the clinicians by a patient.

In some embodiments the ligand or chelating agent that is either covalently or electrostatically bound to the microsphere may include one or more of bisphosphonates, DMSA, DMDTPA, ethylene dicysteine, mercaptoacetyltriglycine, hydrazinonicotinamide, iminodiacetic acid, a crown ether, DTPA monoamide, EDTA, DOTA, EGTA, BAPTA, DO3A, NOTA-Bn, styrene, butyl acrylate, glycidil methacrylate, aminocarboxylic acids, NODASA, NODAGA, peptides, oligomers, amino acids, 10-decanedithiol (HDD), ethyl cysteinate dimer complexes, DEDC, methoxyisobutylisonitrile, or derivatives.

In various embodiments a method of medical treatment of a tumor in a mammal is disclosed. The method includes administering a therapeutically effective amount of the non-iodinated, radiopaque microbead or microsphere composition into the subject. In various embodiments the method is used in image-guided embolization or radioembolization of the tumor. A radioisotope may conjugated to the ceramic material or the prepared microbeads that may render imageable computed tomography (CT) contrast or magnetic resonance imaging (MRI) contrast, or both, when administered to the subject.

In various embodiments the non-iodinated radiopaque microbeads with MRI imageability for transarterial radioembolization can be used in patients with specific allergies toward iodinated contrast agents. Also these microbeads may be used in patients with deranged renal function in whom iodinated contrast cannot be used. Also, CT and MRI imageability allows imaging guided administration of the microbeads that may identify the actual location of the microbeads in the embolized tumor vessel. The CT and MRI imageability may also identify if the microbeads block the tumor vessels or if the microbeads have moved into other adjacent vessels which supply blood to normal structures. Also CT and MRI imageability of the microbeads allow follow up of patients without need for additional use of contrast for imaging guided procedures like radiofrequency ablation, microwave/alcohol ablation.

Further the biodegradability of the microbeads allows retreatment by TARE, using the radiolabelled microbeads in case of recurrent/residual tumors at the initial site. This is possible because the blood vessels are not permanently blocked. Also the damage to normal tissue around the tumor are reduced as branches supplying normal tissue, if accidently blocked will reopen after the beads degrade. The microbeads may be indigenously produced using more affordable materials that makes it more affordable to patients especially in developing countries. Further the microbeads may be radiolabelled in any standard nuclear medicine department hot-lab. This reduces the shipment cost because the unlabelled microbeads do not require any radiation protection measures during shipment. Dosage of radioisotope for radiolabelling can be decided by the doctor on a patient specific basis. Radiopaque microbeads without radiolabeling may also be used for embolization of benign conditions like arteriovenous malformations.

While the invention has been disclosed with reference to certain embodiments, it will be understood by those skilled in the art that various changes may be made and equivalents may be substituted without departing from the scope of the invention. In addition, many modifications may be made to adapt to a particular situation or material the teachings of the invention without departing from its scope. Further, the examples to follow are not to be construed as limiting the scope of the invention which will be as delineated in the claims appended hereto.

### EXAMPLES

### Example 1: Preparation of Molybdenum Doped Calcium Phosphate and Calcium Molybdate

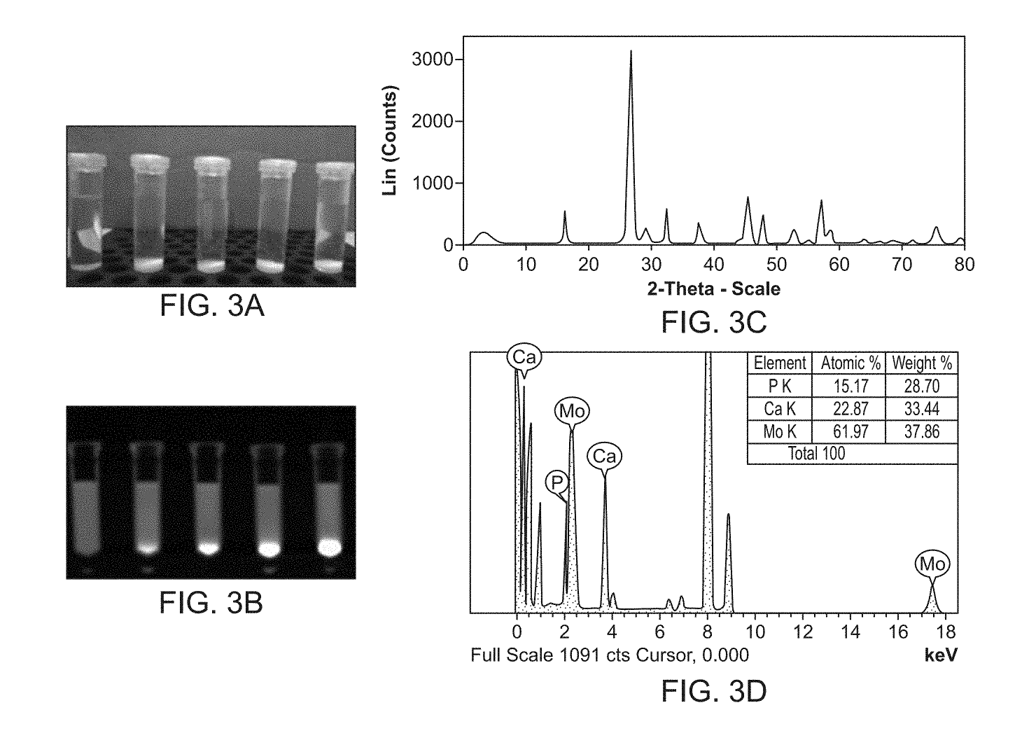

20 ml of 0.5M calcium chloride (Sigma, USA) was mixed with 0.1M ammonium molybdate (Nice Chemicals, India). Volume of ammonium molybdate varied as per required doping percentage (degree of radiopacity). 10 ml of 0.3M diammonium hydrogen phosphate (Fisher Scientific, India) mixed with 10 ml of 3N ammonium hydroxide (Fisher Scientific, India) was added drop-wise to the above mixture shown in FIG. 3A under constant stirring and temperature maintained at 50° C. The precipitate produced was washed 5 times with distilled water. Variation in radiopacity rendered by using different dopant concentrations was identified with CT imaging of the resultant precipitates shown in FIG. 3B. Linear correlation was identified between the concentration of the impurity dopant and radiopacity. Dopant concentration was titrated to provide adequate imaging capability on clinically used CT cameras. Successful doping and chemical composition of the doped ceramics was confirmed using ICP, XRD technique as illustrated in FIG. 3C and EDX techniques as illustrated in FIG. 3D. Biocompatibility of the molybdenum doped calcium phosphate was confirmed in vitro in peripheral blood mononuclear cells and also in vivo in Sprague Dawley (SD) rat models.

### Example 2: Preparation of Calibrated Radiopaque Microbeads

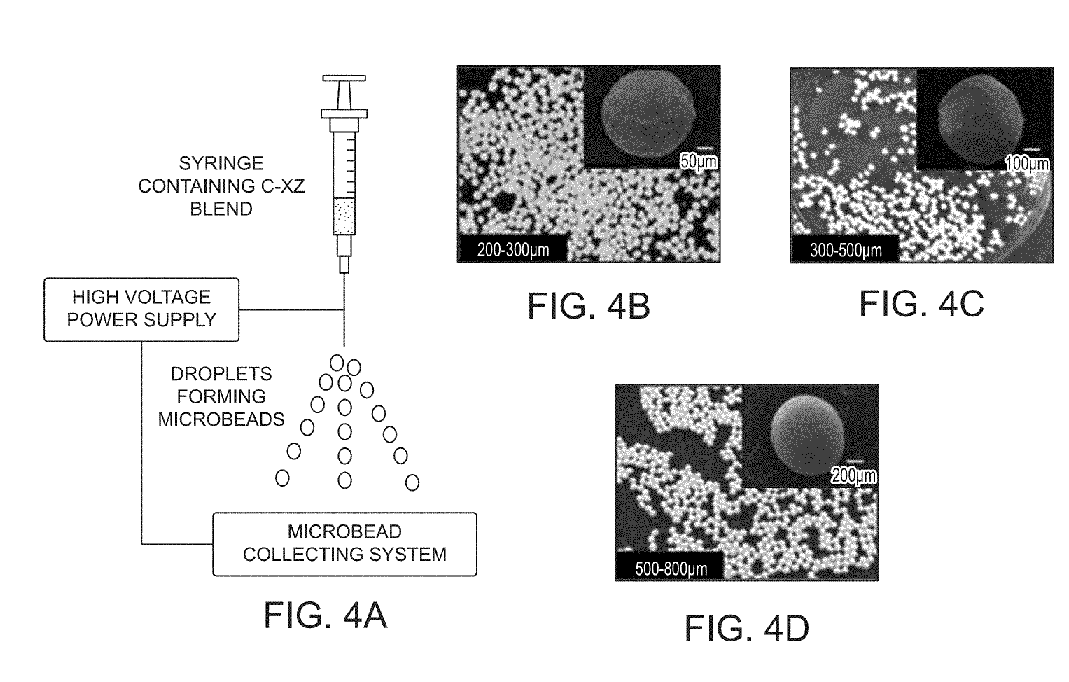

Molybdenum doped (in optimized concentration) calcium phosphate (prepared as described above) was blended with 2% (w/v) sodium alginate solution (in distilled water) in a 4:1 (w/w) concentration. This blend was electrosprayed as illustrated in FIG. 4A into a 2% CaCl2 solution. The electrospraying parameters, such as flow-rate, voltage and height of syringe from the collecting system were optimized to produce microbeads of desired size ranges. The prepared microbeads were washed 5 times in distilled water to remove unreacted reagents and dried in a hot-air oven. Radiopaque microbeads of 3 distinct size ranges 200-300 μm, 300-500 μm and 500-800 μm are shown in FIG. 4B, FIG. 4C and FIG. 4D respectively that were prepared using the technique described. The corresponding scanning electron microscopy (SEM) images (inset) further confirmed their size ranges.

### Example 3: Demonstrating Characteristics of the Radiopaque Microbeads

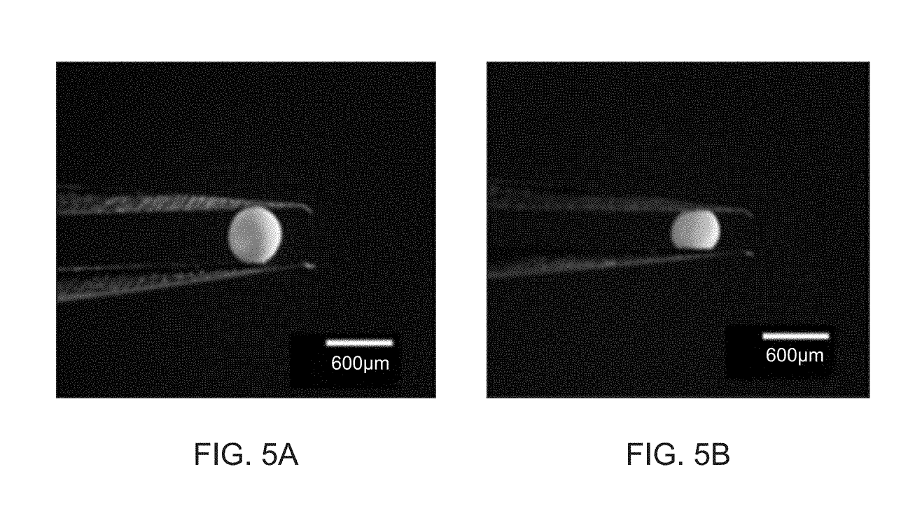

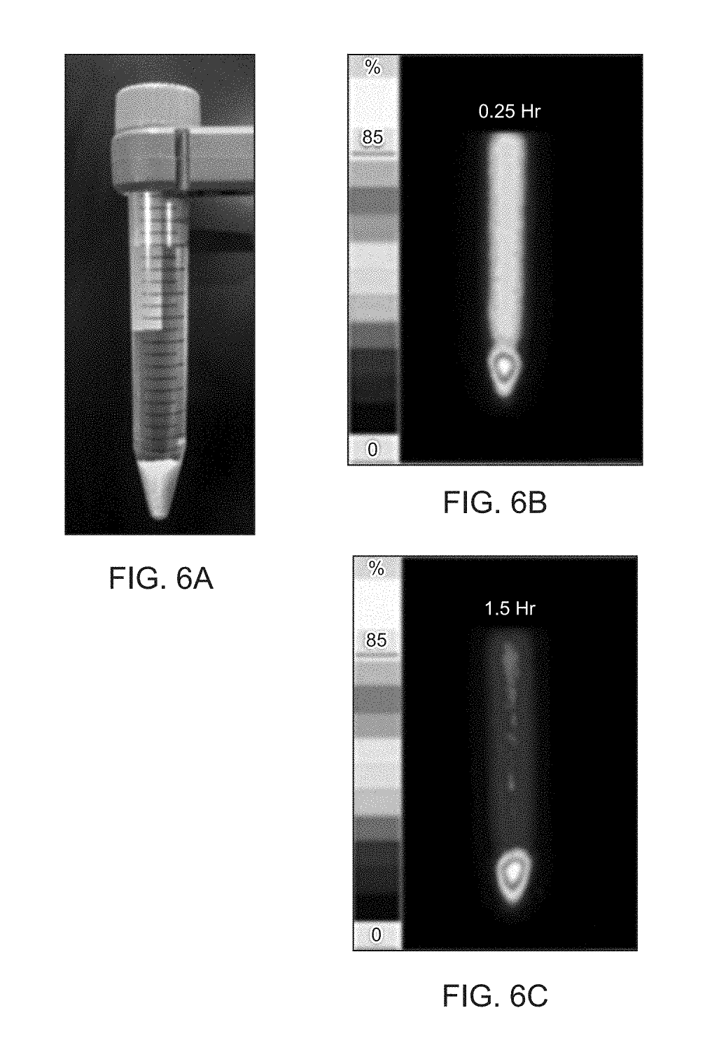

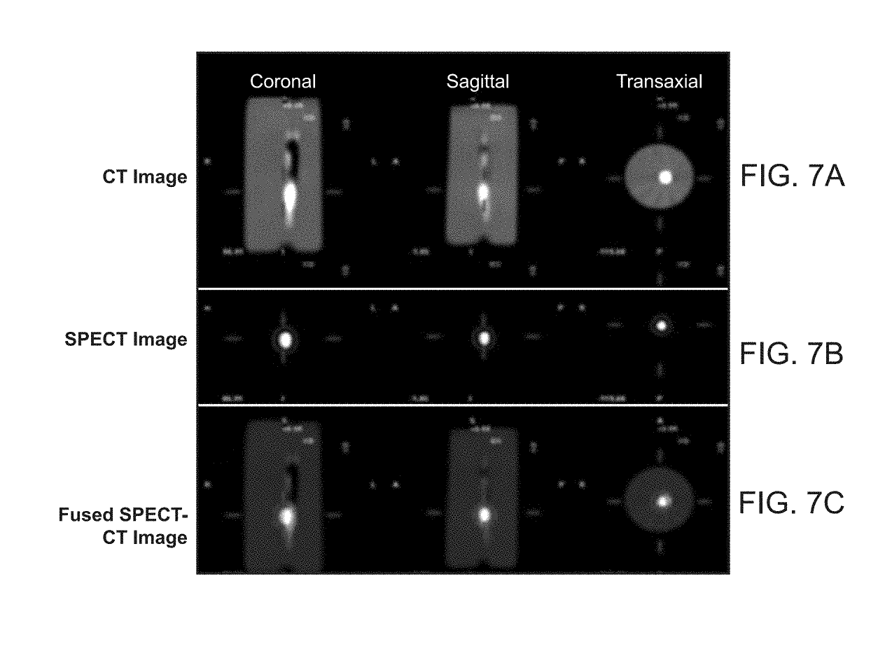

Optical and SEM imaging (FIG. 3) confirmed the spherical shape and size of the microbeads. SEM imaging of cut-sections of the microbeads confirmed their relatively solid structures. FIG. 5A shows that the spherical microbeads, when moist, are pliable and can be compressed to nearly 70% of their initial diameters as shown in FIG. 5B. This allows for their easy passage through small-bore catheters used in the clinics. The radiopaque microbeads were radiolabeled with 99mTc methylene diphosphonate (Tc-MDP) by incubating them as shown in FIG. 6A with the radiopharmaceutical at room temperature and occasional stirring. Serial nuclear imaging of the beads (on a GE dual-head Hawkeye SPECT CT system) and counting the labeled radioactivity on the microbeads (using a Capintec well counter), at regular intervals as shown in FIG. 6B and FIG. 6C the stable and efficient (>85%) radiolabeling ability of the microbeads was identified. These properties (radiopacity and stable radiolabeling) were further confirmed in phantom experiments using agar phantoms as they mimic soft-tissue density. CT, SPECT and fused SPECT-CT images of the agar phantom containing the radiolabeled radiopaque microbeads, as depicted in FIG. 7A, FIG. 7B and FIG. 7C respectively, confirmed the earlier findings.

### Example 4: Demonstration of Degradation of the Radiopaque Microbeads

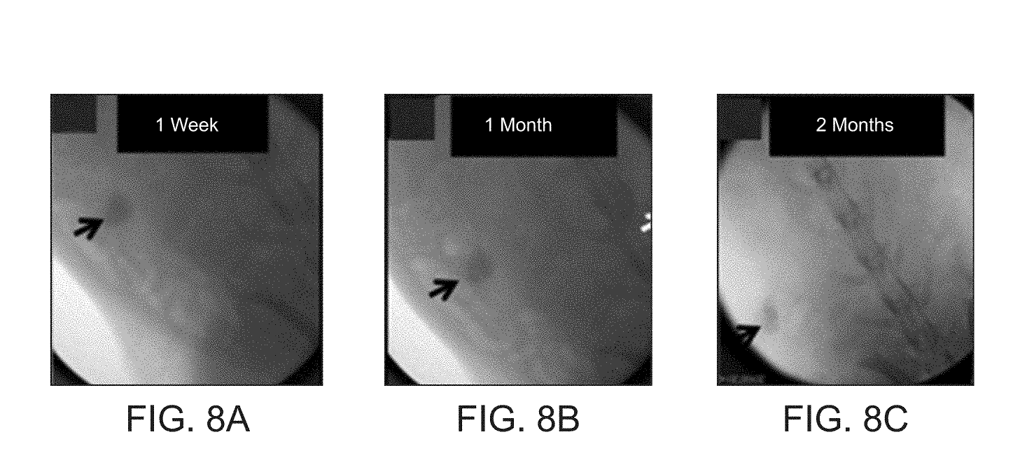

In vitro degradation of the radiopaque microbeads was tested by incubating them in phosphate buffered saline at 37° C. in a shaking incubator. The microbeads showed early signs of degradation after a period of 2 months. In vivo degradation of the microbeads was demonstrated by implanting them subcutaneously in NZW rabbit models and then perform serial imaging (X-ray) at regular intervals to look for reduction in the x-ray attenuation of the microbeads. FIG. 8A, FIG. 8B and FIG. 8C depicts the results of the described experiment, wherein the intensity of x-ray attenuation seen to be intact in the implanted microbeads at 1 week as shown in FIG. 8A and 1 month post implantation as shown in FIG. 8B is reduced in the 2 month image as shown in FIG. 8C. Thus indicating that the radiopaque microbeads begin to degrade in vivo at approximately 2 months.

### Example 5: In Vivo Demonstration of the Embolizing Potential of the Radiopaque Microbeads

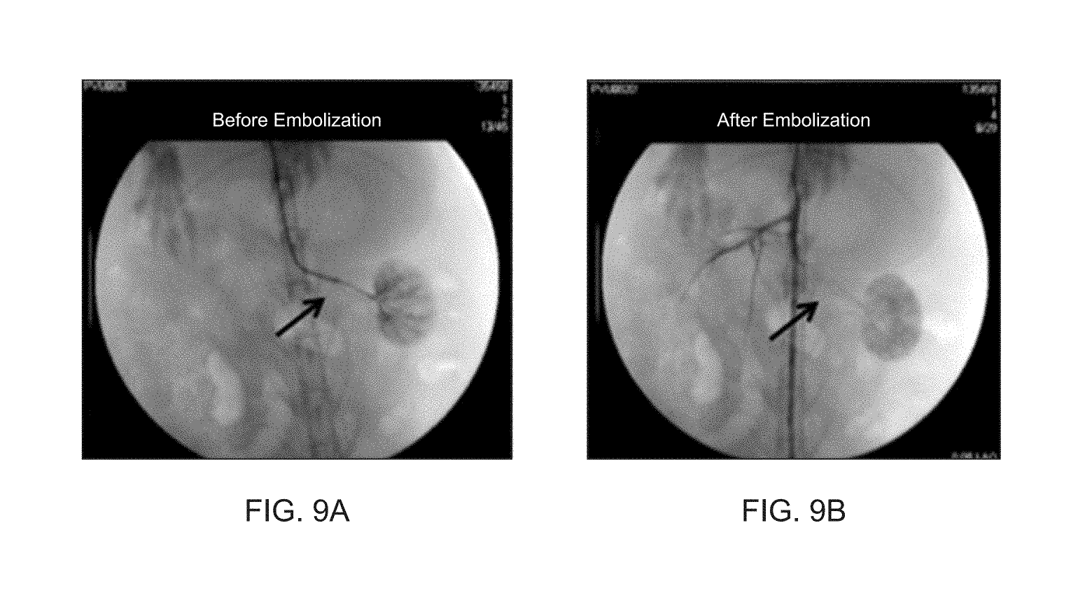

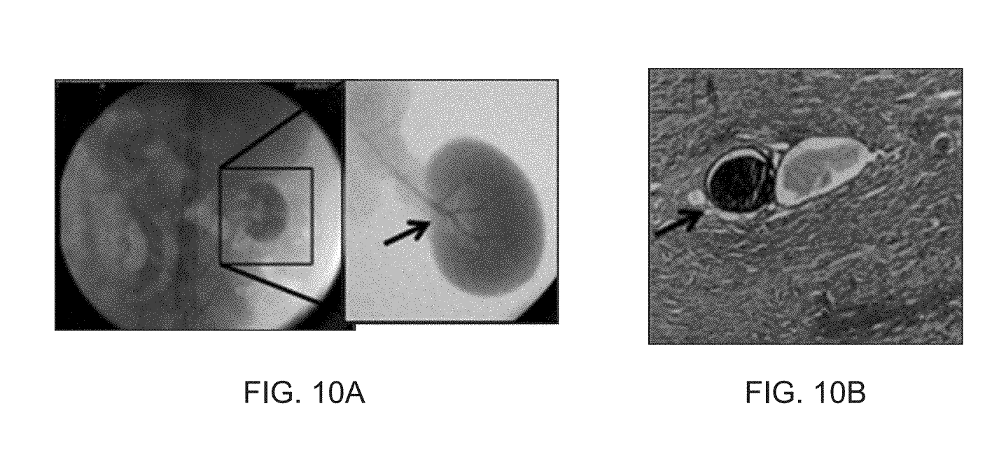

The NZW rabbit renal (kidney) artery embolization model was selected for demonstration of the image guided embolization potential of the radiopaque microbeads. Under image guidance (x-ray C-arm from GE Healthcare) a (clinically used) 4-French arterial catheter was passed in the descending abdominal aorta of the animal via its right carotid artery. The right renal artery was preferentially catheterized and its patency demonstrated by x-ray contrast angiography as shown in FIG. 9A. This was followed by deployment of the radiopaque microbeads into the right renal artery and the subsequent angiogram (of the descending abdominal aorta) showed complete cut-off of the right renal arterial system as shown in FIG. 9B, while the contrast was seen to normally enter the remaining descending aorta and its other branches. X-ray imageability of the microbeads post-embolization was also demonstrated in FIG. 10A. To further prove that the x-ray contrast seen in the embolized right renal arterial system was indeed due to the radiopaque microbeads, the kidney was harvested and subjected to histological evaluation. The h&e stained section of the kidney as shown in FIG. 10B, confirms the presence of the intact radiopaque microbeads in the renal arterial system (black arrow); which were the cause of the embolization of the vessel as well as the radiopacity.

What is claimed is:

1. A non-iodinated, radiopaque microbead or microsphere composition for use in image-guided embolization or radioembolization in a subject, comprising:
a ceramic material (C), comprising an impurity dopant (X), wherein the impurity dopant X is present at a concentration of 0-30% (w/w) and is selected from molybdenum, tungsten, zirconium or gold or a combination thereof;
a polymer binder blended to the ceramic material; and
a radioisotope conjugated to the ceramic material or the microbeads,
wherein the composition renders imageable computed tomography (CT) contrast or magnetic resonance imaging (MRI) contrast, or both, when administered to the subject.

2. The composition of claim 1, wherein the image-guided embolization is selected from transarterial embolization (TE), transarterial radioembolization (TARE) or Selective Internal Radiation Therapy (SIRT) of tumors.

3. The composition of claim 1, wherein the size of the microsphere is 1-1500 μm.

4. The composition of claim 1, further comprising a magnetic resonance imaging (MRI) contrast agent or dopant, doped or loaded in the ceramic material.

5. The composition of claim 4, wherein the MRI contrast agent is selected from iron, manganese, terbium, erbium, dysprosium, holmium, thulium, bismuth, barium, strontium, iodine, zirconium, lanthanides, hafnium or aluminium.

6. The composition of claim 1, wherein the ceramic is selected from the group consisting of alumina, zirconia, silica, hydroxyapatite, calcium aluminate, bioactive glass, cerium oxide, calcium sulphate, calcium molybdate, calcium silicates, calcium carbonate, α-tricalcium phosphate, β-tricalcium phosphate, octocalcium phosphate, dicalcium phosphates, tetracalcium phosphate monoxide, ferric-calcium-phosphorus oxides, biocorals or any combination thereof.

7. The composition of claim 1, wherein the polymer is selected from the group comprising of polyvinyl alcohols, polyacrylic acids, polymethacrylic acids, polyethylenimines, poly vinyl sulphonates, carboxymethyl celluloses, hydroxymethyl celluloses, oxidized cellulose, gellan, gum arabic, substituted celluloses, polyanhydrides, poly (ortho)esters, polyacrylamides, polyethylene glycols, polyamides, polyvinylpyrrolidones, polyureas, polyurethanes, polyesters, polyethers, polystyrenes, polysaccharides, polylactic acids, polyethylenes, polymethyl methacrylates, polycaprolactones, polyvinyl acetate, polyglycolic acids, poly(lactic-co-glycolic) acids, albumin, transferrin, caseins, gelatin, mannose, sucrose, starch, galactose, galactomannans, or a combination thereof.

8. The composition of claim 1, wherein the polymer is a biopolymer selected from alginate, gelatin, collagen, chitosan, carboxymethyl chitosan, chitin, cellulose, carboxymethyl cellulose, dextran, fibrin, hyaluronic acid, chondroitin sulphate, agarose, starch, poly[lactic-co-glycolic] acid, poly-L-lactic acid, polylactic acid, polycaprolactone, polyvinyl alcohol, polyhydroxy butyrate, polyhydroxy butyrate co hydroxyvalerate, polyphosphazenes, polyurethane, or polyanhydrides.

9. The composition of claim 1, wherein the polymer is present at a concentration of 0-30% (w/w) of the ceramic.

10. The composition of claim 1, wherein the microspheres are biodegradable and undergo degradation in at least two months.

11. The composition of claim 1, wherein the composition is administered by intra-arterial, transarterial, intra-articular or local route.

12. The composition of claim 1, wherein the microspheres are non-iodinated and show combinatorial CT and MRI contrast in T2 mode with relaxivity of at least R₂=17 mM⁻¹s⁻¹.

13. The composition of claim 1, the radioisotope is selected from ^{99m}Tc, ¹¹¹In, ¹²³I, ¹³¹I, ¹⁸⁸Re, ¹⁸⁶Re, ¹⁶⁶Ho, ³²P, ¹⁸F, ⁶⁸Ga, ¹⁷⁷Lu, ⁹⁰Y, ¹⁶⁶Dy, ¹⁰³Pd, ¹⁶⁹Yb, ²¹²Bi, ²¹³Bi, ²¹²Po, ²²⁵Ac, ²¹¹At, ⁸⁹Sr, ¹⁹²Ir, ¹⁹⁴Ir or ²²³Ra conjugated to the microspheres.

14. A method of synthesis of radiopaque microsphere or microbeads capable of rendering an imageable computed tomography (CT) contrast when administered in a subject comprising:
doping or co-loading an impurity dopant in a ceramic to obtain a ceramic material, wherein the impurity dopant is present at a concentration of 0-30% (w/w) and is selected from molybdenum, tungsten or zirconium or a combination thereof, for X ray contrast and from iron, manganese, terbium, erbium, dysprosium, holmium, thulium, bismuth, barium, strontium, iodine, zirconium, lanthanides, hafnium or aluminium or thereof, for MRI contrast;
blending the ceramic material with a polymer solution;
electrospraying the blend with or without a crosslinker to form microbeads;
incubating the formed microbeads at an optimum temperature for 48 hours and
radiolabeling the microbeads with a radioisotope to produce the radiolabeled radiopaque microbeads.

15. The method of claim 14 wherein the crosslinker is selected from one of bivalent cationic solutions comprising calcium chloride, stannous chloride, barium chloride or ferric chloride, carbodiamides, EDC, trivinyl sulphones, acrylamides, epoxides, polyamides, maleimide, iminoesters, or combinations thereof.

16. The method of claim 14 further comprising the step of calcining the beads in the temperature range 100-1000° C. to produce the radiopaque beads.

17. The method of claim 14 wherein the beads are further subjected to lyophilization to produce the radiopaque beads.

18. The method of claim 14, wherein the synthesized microbeads are non-iodinated

19. The method of claim 14, wherein the radiolabeling of microbeads is done either by direct interaction or by using an appropriate ligand or chelating agent.

20. The method of claim 19 wherein, the ligand or chelating agent is selected from bisphosphonates, DMSA, DMDTPA, ethylene dicysteine, mercaptoacetyltriglycine, hydrazinonicotinamide, iminodiacetic acid, a crown ether, DTPA monoamide, EDTA, DOTA, EGTA, BAPTA, DO3A, NOTA-Bn, styrene, butyl acrylate, glycidil methacrylate, aminocarboxylic acids, NODASA, NODAGA, peptides, oligomers, amino acids, 10-decanedithiol (HDD), ethyl cysteinate dimer complexes, DEDC, methoxyisobutylisonitrile, or derivatives or combinations thereof.

21. The method of claim 19, wherein the ligand or chelating agent, with or without radiolabeling, is either covalently or electrostatically bound to the microbeads.

22. The method of claim 14, further comprising conjugating the microsphere with a chemotherapeutic agent.

23. A method of medical treatment of a tumor in a mammal comprising:
administering a therapeutically effective amount of a non-iodinated, radiopaque microbead or microsphere composition for use in image-guided embolization or radioembolization of the tumor, comprising:
a ceramic material (C), comprising an impurity dopant (X), wherein the impurity dopant is present at a concentration of 0-30% (w/w) and is selected from molybdenum, tungsten, zirconium or gold or a combination thereof;
a polymer binder blended to the ceramic material, and
a radioisotope conjugated to the ceramic material or the prepared microbeads, wherein the composition renders imageable computed tomography (CT) contrast or magnetic resonance imaging (MRI) contrast, or both, when administered to the subject.

24. The method of claim 23 wherein the non-iodinated radiopaque microbeads are used in combination with radiofrequency ablation, chemotherapy or immunotherapy.

25. The method of claim 23 wherein the non-iodinated radiopaque microbeads are biodegradable.

26. The method of claim 23 wherein the method is repeated on re-occurrence of the tumor.
